(19) United States
(12) Patent Application Publication
(10) Pub. No.: US 2004/0224336 A1
(43) Pub. Date: Nov. 11, 2004

(54) RecA-assisted specific oligonucleotide extension method for detecting mutations, SNPs and specific sequences

(75) Inventor: Robert E. Wagner, Carr, CO (US)

Correspondence Address: VENABLE, BAETJER, HOWARD AND CIVILETTI, LLP, P.O. BOX 34385, WASHINGTON, DC, 20043-9998, US

(73) Assignee: GENE CHECK, INC., Fort Collins, CO

(21) Appl. No.: 10/792,785

(22) Filed: Mar. 5, 2004

Related U.S. Application Data
(60) Provisional application No. 60/453,640, filed on Mar. 11, 2003.

(51) Int. Cl. C12Q 1/68

(52) U.S. Cl. 435/6

(57) ABSTRACT

A method for detecting a specific sequence, a mutation and/or a polymorphisms, including a SNP, is based on the use RecA or a RecA-like recombinase protein and the process of allele specific oligonucleotide extension. RecA coated, specific DNA oligonucleotide probes (RecA filaments) are used for homology searching in duplex DNA. Location of homologous sequences results in the formation of D-loop or double D-loop structures containing a duplex regions comprising the oligonucleotide probe and one strand of the target DNA. Probes are selected to terminate with their 3′ end at the site of the mutation or the SNP, such that extension depends on correct nucleotide pairing, which occurs only when the probe is annealed to a target DNA which comprises the allele complementary to the 3′ end of the probe. Successful extension is diagnostic of the specific sequence, mutation or SNP. Also provided are compositions and kits useful for practicing the above methods.

### BACKGROUND OF THE INVENTION

1. Field of the Invention

The present invention in the fields of molecular biology and medicine relates to methods for detecting specific sequences in double-stranded DNA samples and for detecting mutations and polymorphisms involving as little as one base change (Single Nucleotide Polymorphism—SNP) or additions to or deletions from the wild-type DNA sequence.

2. Description of the Background Art

Progress in human molecular and medical genetics depends on the efficient and accurate detection of mutations and sequence polymorphisms, the vast majority of which results from single base substitutions (Single Nucleotide Polymorphisms or SNPs) and small additions or deletions. Assays capable of detecting the presence of a particular mutation, a SNP or a mutant nucleic acid sequence in a sample are therefore of substantial importance in the prediction and diagnosis of disease, forensic medicine, epidemiology and public health. Such assays can be used, for example, to detect the presence of a mutant gene in an individual, allowing determination of the probability that the individual will suffer from a genetic disease, and to detect the presence of an infectious agent in a patient. The ability to detect a mutation has taken on increasing importance in early detection of cancer or discovery of susceptibility to cancer with the discovery that discrete mutations in cellular oncogenes can result in activation of that oncogene leading to the transformation of that cell into a cancer cell and that mutations inactivating tumor suppressor genes are required steps in the process of tumorigenesis The detection of SNPs has assumed increased importance in the identification and localization (mapping) of genes, including those associated with human and animal diseases. Further, the continuing and dramatic increase in the number of SNPs of known location in the genome will allow genome wide scanning for identification of disease associated genes and help usher in the era of personalized medicine.

To realize the maximum potential benefits of this explosion of genetic information, both in research and in health care applications, and to increase the utility and applicability of mutation and SNP detection will require improvements in current technologies, including increases in assay sensitivity and multiplexing ability and reductions in assay complexity and cost. The present invention is directed to methods of specific sequence, SNP and mutation detection embodying such improvements.

Most methods devised to attempt to detect genetic alterations comprising one or a few bases involve amplification of specific DNA regions by polymerase chain reaction (PCR). However, PCR amplification has severe limitations with respect to its utility in mutation and SNP detection:

1. PCR amplification is a relatively low fidelity process. Misincorporation during amplification is a particular problem in those detection methods that involve denaturation and annealing of PCR amplicons to form mutant: wild type heteroduplexes in which mutations and SNPs are revealed as mismatched or unpaired bases. Given the random nature of PCR errors, virtually all will be in such mismatches following annealing and will contribute to background signal. In gel based applications these error-containing molecules will generally not interfere. However, in high through put applications involving mismatch binding or mismatch cleaving, high background signals can greatly limit the utility of a method and frequently require that PCR fragments be kept relatively short.

2. PCR is subject to mispriming. Mispriming involves primer extension at non-target sites, which can occur even when only a relatively short portion of the 3′ end of a primer is transiently paired with some sequence in the target DNA. Mispriming can produce long single-stranded fragments which can adopt mismatch-containing secondary structure. Mispriming is also a major problem in those methods which utilize primer extension for SNP detection. These methods use oligonucleotides which are complementary to a region of target DNA immediately adjacent to the SNP or mutation to be genotyped such that the first nucleotide added by DNA polymerase to the 3′ end of the oligonucleotide will be complementary to and diagnostic for the SNP. Generally, these methods use specific nucleotide terminators (e.g., dideoxy or acyclo nucleotides) which are detectably labeled. Mispriming is such a problem with these methods that they generally require preamplification of the target region.

3. Some DNA regions are refractory to amplification. Because PCR requires denaturation of the target DNA, it provides the opportunity for the target DNA to adopt secondary structures, some of which may prevent primer annealing or extension.

4. PCR multiplexing potential is limited. The intricacies of primer design and the variability of PCR conditions depending on target and primer sequences coupled with the potential for interference between primer sets makes it unlikely that PCR will ever attain multiplexing levels as high as 100 fold, levels generally considered as the minimum desirable level for high through put SNP and mutation detection applications.

A method of mutation/SNP that is not dependent on PCR amplification would have immediate and widespread utility both in research and healthcare. The present invention does not require PCR amplification.

Allele Specific Amplification

Allele specific amplification (Newton et al., U.S. Pat. No. 5,595,890) is a method of PCR amplification that selectively amplifies only one allele of a given SNP or mutation. The method involves selecting one PCR primer (diagnostic primer) that is substantially complementary to the target DNA except at the 3′ end where “a 3′ terminal nucleotide of the diagnostic primer [is] either complementary to a suspected variant nucleotide or to the corresponding normal nucleotide.” An extension product is obtained only when the terminal nucleotide is complementary to the corresponding nucleotide in the target DNA sequence and is revealed by amplification using a second, amplifying primer.

Allele specific amplification, in contrast with the present invention, requires an amplification primer, denaturation of the target DNA to allow hybridization of the diagnostic and amplification primers and is clearly dependent on PCR for detection. Further, complete genotyping requires separate amplification reaction with diagnostic primers with 3′ termini complementary to each of the alleles of the SNP or mutation in question. Simultaneous exposure of a target DNA sample to both diagnostic primers (in the case of a two allele SNP) will always give an amplification product and will not allow genotyping unless an additional step, such as gel electrophoresis or mass spectroscopy is included. For the products to be distinguishable, the diagnostic primers must be sufficiently different, i.e., different in length or containing different adducts, such that the amplification products can be separated and distinguished by some means.

RecA

RecA is a bacterial protein involved in DNA repair and genetic recombination and has been best characterized in E. coli. RecA is the key player in the process of genetic recombination, in particular in the search and recognition of sequence homology and the initial strand exchange process. RecA can catalyze strand exchange in the test tube. Recombination is initiated when multiple RecA molecules coat a stretch of single-stranded DNA (ssDNA) to form what is known as a RecA filament. This filament, in the presence of ATP, searches for homologous sequences in double-stranded DNA (dsDNA). When homology is located, a three stranded (D-loop) structure is formed wherein the RecA filament DNA is paired with the complementary strand of the duplex.

RecA homology searching is extremely precise and RecA has been used to facilitate screening of plasmid libraries for plasmids containing specific sequences (Rigas et al., Proc Natl Acad Sci USA. 83:9591-9595 (1986)). In this application, biotinylated ssDNA probes are reacted with RecA to form RecA filaments. The filaments are used for homology searching in circular plasmid DNA. When the probes are removed by binding to avidin, those plasmids containing sequences homologous to the probes are isolated by virtue of the triple stranded (D-loop) structures formed by the RecA filament and the plasmid duplex. In order for these structures to be stable it is necessary to use adenosine 5′-[γ-thio]triphosphate (ATP[γ-S]) in place of ATP. ATP[γ-S] allows homology searching by RecA, by is non-hydrolyzable and thus does not allow RecA dissociation from the triple stranded structure.

RecA has also been used, in a variety of applications, to facilitate the mapping and/or isolation of specific DNA regions from bacterial and human genomic DNA (Ferrin, L J, et al., Science 254:1494-1497 (1991); Ferrin, L J, et al., Nature Genetics 6:379-383 (1994); Ferrin, L J and Camerini-Otero, R D, Proc Natl Acad Sci 95:2152-2157 (1998), Sena et al., U.S. Pat. Nos. 5,273,881 and 5,670,316; Sena and Zarling, Nature Genetics 3:365-371 (1993)). In one of these applications (Ferrin, L J, et al., Science 254:1494-1497 (1991); Ferrin et al., U.S. Pat. No. 5,707,811; Ferrin, L J, et al., Nature Genetics 6:379-383 (1994)), RecA is used in conjunction with restriction enzymes (sequence specific double strand DNA endonucleases) to allow isolation or identification of specific DNA fragments. RecA filaments are prepared and reacted with genomic DNA under conditions that allow triple strand (D-loop) structure formation. The DNA is then treated with either a restriction endonuclease or a modification methylase (methylase action transfers a methyl group to the specific recognition sequence of a specific restriction endonuclease, thus protecting the sequence from endonuclease digestion). The presence of the RecA filament in the triple strand structure prevents digestion or methylation.

In a more recently developed application (Ferrin et al., U.S. Pat. No. 5,707,811; Ferrin, L J and Camerini-Otero, R D, Proc Natl Acad Sci 95:2152-2157 (1998)), specific RecA filaments have been used to protect restriction endonuclease generated “sticky ends” from being filled in by DNA polymerase such that, upon removal of the RecA filaments, specific fragments can be cloned into plasmid vectors. In this application, genomic DNA is digested with one or more restriction enzymes that produce recessed 3′ ends. A specific fragment from this digestion is protected by triple strand structure formation with a pair of RecA filaments. The recessed 3′ ends of the remaining fragments are then filled in with a polymerase. The polymerase is removed or inactivated, the RecA, filament is removed and the specific fragment cloned by virtue of its sticky ends.

RecA has been used in association with DNA ligase to label specific DNA fragments (Fujiwara, J et al., Nucl Acids Res 26:5728-5733 (1998)). In this application, oligonucleotides are designed to allow the 3′ end to form a double-stranded region by folding back on a portion of itself (hairpin), RecA is then used to coat the remaining single-stranded 3′ region and the resulting RecA filament used to perform homology searching. When a terminus of the target DNA is complementary to the single-stranded portion of the oligonucleotide, ligation can covalently link the oligonucleotide, which can be labeled at the 5′ end with a detectable label, to the target DNA to allow detection or isolation of specific target DNA sequences without denaturation of the target DNA.

Formation of RecA catalyzed double D-loops has been used to identify and isolate specific DNA regions from dsDNA (Sena et al., U.S. Pat. Nos. 5,273,881 and 5,670,316; Sena and Zarling, Nature Genetics 3:365-371 (1993)). This method requires relatively long DNA probes (>78 nucleotides), complementarity between the probes and double D-loops in order to provide for a stable structure. These documents note the possibility of introducing a detectable label into the probe by oligonucleotide extension with DNA polymerase. Importantly, this method is only suited for detection of specific sequences in a target DNA but is of no use in detecting mutations or SNPs a primary objective of the present invention.

No uses of RecA, other than those disclosed in the commonly assigned U.S. patent applications of the present inventor and colleague (U.S. Ser. No. 10/078,278; and U.S. Ser. No. 10/283,243), have heretofore been proposed to allow the detection of mutations or SNPs or the identification of sequences which differ from a wild type sequences by only one or a few nucleotides.

### SUMMARY OF THE INVENTION

The present invention is directed to a RecA assisted method for detecting of a mutation and/or a SNP or of a specific DNA sequence in a double-stranded target or test DNA molecule, which will hereinafter be referred to as the RecA/Allele specific oligonucleotide extension (RecA/ASOE) method.

The RecA/ASOE method of SNP and mutation detection comprises:

(a) providing a ssDNA probe which is optionally detectably labeled or which optionally includes an adduct at its 5′ end or internally that allows immobilization, which probe has a known nucleotide sequence complementary to the sequence of at least a part of the target DNA, the sequence of which is such that, when annealed to the complementary region of the target DNA, the 3′ end of the probe covers the site of the mutation or SNP and is complementary to one allele of the mutation or SNP;

(b) contacting the probe with a RecA protein (or a homologue thereof, defined in more detail below) to form a RecA filament;

(c) contacting the RecA filament with target dsDNA, thereby allowing RecA filament homology searching which leads to the formation of a three stranded DNA D-loop structure in the target DNA. The D-loop structure comprises the probe and the two strands of the target DNA;

(d) contacting the DNA D-loop structure, in the presence deoxyribonucleotide triphosphates (dNTPs), which may optionally be detectably labeled or include an adduct which allows immobilization, with a DNA polymerase capable of primer extension;

(e) allowing extension of the probe, wherein extension depends on the correct base pairing of the 3′ end of the probe with the target SNP, mutation or specific sequence; and

(f) detecting the extension, i.e., the presence of the dNTPs covalently attached to the 3′ end of the DNA probe. Extension of the probe is indicative of the presence of the specific allele of the mutation or SNP in the target DNA.

Also provided is a method for detecting specific sequences in a sample of double-stranded target or test DNA, for example, DNA of an infectious viral or bacterial agent in a sample of mammalian genomic DNA, wherein D-loop formation and consequent probe extension are dependent upon the presence, in the target DNA sample, of the specific sequence.

The RecA/ASOE method for detecting a specific sequence comprises:

(a) providing a ssDNA probe which is optionally detectably labeled or which includes an adduct at its 5′ end or internally to allow immobilization, which probe has a known nucleotide sequence complementary to a specific DNA sequence;

(b) contacting the probe with a RecA protein or homologue to form a RecA filament;

(c) contacting the RecA filament with target dsDNA, wherein RecA filament homology searching and the presence in the target DNA sample of sequence complementary to the probe sequence allows formation of a three stranded DNA D-loop structure in the target DNA;

(d) contacting the DNA D-loop structure, in the presence dNTPs, which may optionally be detectably labeled or include an adduct which allows immobilization, with a DNA polymerase capable of primer extension under conditions wherein the oligonucleotide will be extended if and only if the 3′ end of the oligonucleotide is correctly base paired with the target DNA;

(e) detecting the presence of the dNTPs covalently bonded to the 3′ end of the DNA probe, wherein the presence of the dNTPs is indicative of the presence of the specific DNA sequence in the target DNA sample.

The probe may be any ssDNA, including, but not limited to, synthetic oligonucleotides of any length, denatured PCR amplicons and denatured restriction enzyme digestion fragments from any plasmid, viral, bacterial or eukaryotic genomic DNA. Probes are preferably synthetic oligonucleotides 20-120 nucleotides in length, more preferably 40-60 nucleotides in length.

The RecA protein is preferably from E. coli.

In the methods described herein, the labels may be any suitable detectable label, e.g., a fluorophore, a chromophore, a radionuclide, biotin, digoxigenin, etc. The probe DNAs, dNTPs or terminators may be directly labeled by direct bonding or binding of the label. However, the term “detectably labeled,” includes “indirect” labeling wherein the “detectable label” is a primary antibody, or any other binding partner, which is directly labeled. Alternatively, the detectable label is a combination of an unlabeled primary antibody with a directly labeled secondary antibody specific for the primary antibody.

In the present method, probe DNA may be in solution or immobilized to any solid support and may be immobilized either before or after reaction with RecA and target DNA.

In the above methods, the single DNA D-loop structure may be further stabilized by the addition, before step (d) above of the single strand DNA binding (SSB) protein (Chase et al., Nucl Acids Res 8:3215-3227 (1980)), or an SSB homologue.

In the above methods of SNP and mutation detection, stability of the three stranded structure can also be enhanced by utilizing a DNA oligonucleotide complementary to the opposite strand of the target DNA to which the probe or probes are complementary. In this case, the oligonucleotide must contain a nucleotide at the site of the mutation or SNP which is not complementary to any allele of the mutation or SNP.

The present invention also provides a kit useful for detecting a one or more mutations or polymorphisms in a DNA sample or for detecting a specific sequence in a test DNA sample, the kit being adapted to receive therein one or more containers, the kit comprising:

(a) a first container containing RecA protein;

(b) a second container containing DNA probes; and optionally

(c) a third container or plurality of containers containing buffers and reagent or reagents including dNTPs and a DNA polymerase capable of extending DNA probes when the probes are annealed to target DNA.

Also included is a kit useful for detecting a specific mutation or polymorphism or a specific sequence in a DNA sample, the kit being adapted to receive therein one or more containers, the kit comprising:

(a) a first container containing RecA filaments, the filaments comprising RecA protein, or a homologue thereof, and ssDNA probes;

(b) a second container or plurality of containers containing buffers and reagent or reagents including dNTPs and a DNA polymerase capable of extending DNA probes when the probes are annealed to the target DNA.

### BRIEF DESCRIPTION OF THE DRAWINGS

FIGS. 1 and 2 are schematic representations of the RecA/ASOE detection method.

FIG. 1 shows the RecA/ASOE method using a single allele specific probe. The oligonucleotide “probe” is mixed with RecA protein. RecA coats the probe to form a “RecA filament.” RecA filament is added to target DNA and allowed to perform homology searching and to form a triple stranded or “D-loop” structure. A DNA polymerase is added along with dNPTs. If the probe is complementary to the SNP, mutation or specific sequence, i.e., the 3′ end of the probe is base paired, the polymerase will extent the probe by adding nucleotides to its 3′ end. Cycling involves displacement of the original oligonucleotide probe, either before or because of a second round of homology searching by a RecA filament.

FIG. 2 shows the RecA/ASOE method employing a pair of single stranded probes, i.e., the double D-loop method. Oligonucleotide probes are mixed with RecA protein. RecA coats the probes to form RecA filaments. The RecA filaments are added to target DNA and allowed to perform homology searching. If the 3′ ends of the probes are complementary to the SNP, mutation or specific sequence, polymerase will extend them to form a four stranded or “double D-loop” structure. The stability of the double D-loop structure will normally require further homology searching to release the extended fragments, which will allow exponential signal amplification.

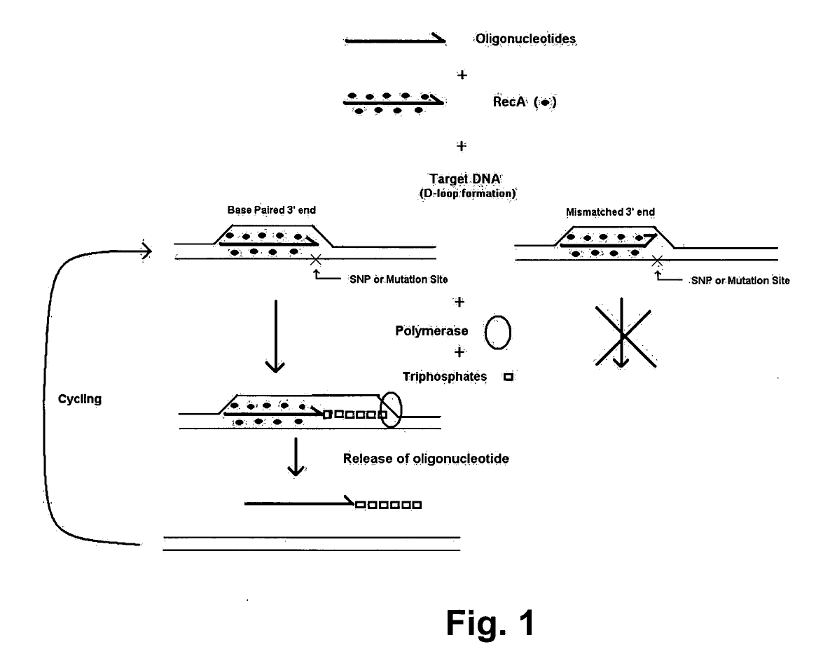

### DESCRIPTION OF THE PREFERRED EMBODIMENTS

The present inventor has devised a novel technology for detecting mutations or SNPs or for detecting specific sequences in dsDNA samples using RecA mediated homology searching followed by genotype or sequence specific oligonucleotide extension (RecA/ASOE). In general, the method employs:

(1) a double-stranded target or test DNA molecule, which may be any synthetic, viral, plasmid, prokaryotic or eukaryotic DNA from any source, including, but not limited to, genomic DNA, restriction digestion fragments or DNA amplified by PCR or any other means;

(2) ssDNA probes, which might be any synthetic oligonucleotide, PCR amplicon, plasmid DNA, viral DNA, bacterial DNA or any other DNA of known sequence or of sequence complementary to the target DNA or to a portion thereof,

(3) E. coli RecA or a homologue thereof, as defined below.

As used herein and in the present claims (for the sake of brevity and clarity), the “RecA” or “SSB” is intended to include either the native or mutant E. coli RecA or SSB protein, or a “homologue” thereof as defined below. A “homologue” of RecA, SSB, etc., is a protein that has functional and, preferably, also structural similarity to its “reference” protein. One type of homologue is encoded by a homologous gene from another species of the same genus or even from other genera. As described below, these proteins, originally discovered in bacteria, have eukaryotic homologues in groups ranging from yeast to mammals. A functional homologue must possess the biochemical and biological activity of its reference protein, particularly the DNA binding selectivity or specificity so that it has the utility described herein. In view of this functional characterization, use of homologues of E. coli RecA or SSB proteins, including proteins not yet discovered, fall within the scope of the invention if these proteins have sequence similarity and the described DNA binding or biological activity or “improved” binding activity. Nonlimiting examples of improvements include a RecA homologue that binds to shorter DNA molecules or an SSB homologue with higher binding affinity for ssDNA.

“Homologues” is also intended to include those proteins which have been altered by mutagenesis or recombination that have been performed to improve the protein's desired function. These approaches are generally well described and well referenced below. Mutagenesis of a protein gene, conventional in the art, is generally accomplished in vivo by cloning the gene into bacterial vectors and duplicating it in cells under mutagenic conditions, e.g., in the presence of mutagenic nucleotide analogs and/or under conditions in which mismatch repair is deficient. Mutagenesis in vitro, also well-known in the art, generally employs error-prone PCR wherein the desired gene is amplified under conditions (nucleotide analogues, biased triphosphate pools, etc.) that favor misincorporation by the PCR polymerase. PCR products are then cloned into expression vectors and the resulting proteins examined for function in bacterial cells.

Recombination generally involves mixing homologous genes from different species, allowing them to recombine, frequently under mutagenic conditions, and selecting or screening for improved function of the proteins from the recombined genes. This recombination may be accomplished in vivo, most commonly in bacterial cells under mismatch repair-deficient conditions which allow recombination between diverged sequences and also increase the generation of mutations. Radman et al. have developed such methods of protein “evolution” (U.S. Pat. Nos. 5,912,119 and 5,965,415). In addition, Stemmer and colleagues have devised methods for both in vivo and in vitro recombination of diverged sequences to create “improved” proteins. Most involve PCR “shuffling” wherein two PCR amplicons of diverged sequences are digested and mixed together such that the fragments serve as both primer and template for additional PCR and, in so doing, combine different segments of the diverged genes, which is, in effect, genetic “recombination.” Frequently, error prone PCR conditions are included to further stimulate generation of novel sequences. Resulting PCR products are cloned into expression vectors, and the resulting proteins are screened for improved function. See, for example, U.S. Pat. Nos. 5,512,463; 5,605,793; 5,81,238; 5,830,721; 5,837,458; 6,096,548; 6,117,679; 6,132,970; 6,165,793; 6,180,406; 6,251,674; 6,277,638; 6,287,861; 6,287,862; 6,291,242; 6,297,053; 6,303,344; 6,309,883; 6,319,713; 6,319,714; 6,323,030; 6,326,204; 6,335,160; 6,344,356, all of which are incorporated by reference.

Thus, a preferred homologue of an E. coli RecA protein or an E. coli SSB protein has (a) the functional activity of native E. coli RecA or SSB and also preferably shares (b) a sequence similarity to the native E. coli protein of at least about 20% (at the amino acid level), preferably at least about 40%, more preferably at least about 60%, even more preferably at least about 70%, even more preferably at least about 80%, and even more preferably at least about 90%

At least 65 RecA genes from different bacteria have been cloned and sequenced (Sandler, S J, et al., Nucl Acids Res 24:2125-2132 (1996); Roca, A I, et al., Crit Rev Biochem Mol Biol 25:415-456 (1990); Eisen, J A, J. Mol. Evol. 41:1105-1123 (1995); Lloyd, A T, et al., J. Mol. Evol. 37:399-407 (1993)). RecA homologues, known as RadA proteins (and genes), have been identified in three archaean species (Sandler et al., supra; Seitz, E M, et al., Genes Dev. 12:1248-1253 (1998)). Eukaryotic homologues of RecA have been identified in every eukaryotic species examined; the prototype eukaryotic RecA homologue is the yeast Rad51 protein (Seitz et al., supra; Bianco, P R, et al., Frontiers Biosci. 3:570-603 (1998)). Therefore, any homologue of E. coli RecA which, like the E. coli protein, forms DNA filaments for initiation of genetic recombination as well as any functional form that has been mutated or evolved in vivo or in vitro is included within the scope of the present invention.

RecA functions in vitro, forming a three stranded structure involving oligonucleotides along sequence stretches as short as 15 nucleotides (Ferrin et al., 1991, supra). Combining the activities of RecA with genotype- or sequence-specific primer extension or oligonucleotide ligation creates a most powerful detection system for mutations/SNPs or specific sequences in which RecA-coated ss DNA catalyzes formation of a three strand (single D-loop) or four strand (double D-loop) structure without the need for prior denaturation of the test dsDNA.

In one preferred embodiment, the present system employs:

(1) RecA;

(2) specific probe oligonucleotides that contain 5′ or internal adducts to allow detection or immobilization; and

(3) DNA polymerase and dNTPs for extension of annealed oligonucleotides, all or some of which dNTPs may be detectably labeled or contain adducts to allow immobilization.

Probe specificity derives from probe sequence. An oligonucleotide probe is designed to be complementary to the target DNA in the specific sequence of interest or to have its 3′ end complementary to a specific allele of a mutation or SNP.

Formation or stabilization of the D-loop formed by the RecA filaments and target DNA may be further enhanced by the addition of single strand binding (SSB) protein from E. coli or a homologue of SSB or by allowing double D-loop formation using an oligonucleotide complementary to the strand opposite that to which the oligonucleotide probe is complementary. When using an oligonucleotide in mutation or SNP detection to stabilize a single D-loop by forming a double D-loop, the stabilizing oligonucleotide must either terminate before the SNP or mutation site or must have a nucleotide at the site of the mutation or SNP that is not complementary to any allele of the mutation or SNP to prevent probe annealing and extension.

In the RecA/ASOE method, detection of mutations, SNPs and specific sequences is accomplished by detecting the covalent linkage (by DNA polymerase) of dNTPs to the oligonucleotide probe molecule.

The DNA oligonucleotide probe may be of any length but is preferably a synthetic oligonucleotide, of about 30-60 bases in length and is specific for a genetic region that is being examined for the presence of a mutation or SNP or for its presence in a particular target DNA sample.

The target DNA may be of any length (up to an entire chromosome) and can be either genomic or plasmid DNA or a PCR amplicon.

The oligonucleotides and/or dNTPs can be directly labeled with fluorophores or fluorescent labels, including, but not limited to, Fluorescein (and derivatives), 6-Fain, Hex, Tetramethylrhodamine, cyanine-5, CY-3, allophycocyanin, Lucifer yellow CF, Texas Red, Rhodamine, Tamra, Rox, Dabcyl.

RecA filament formation can be accomplished, for example, in a Tris-HCl or Tris-acetate buffer, (20-40 mM, pH 7.4-7.9) with MgCl2 or Mg acetate (1-4 mM), dithiothreitol (0.2-0.5 mM), and ATP or ATP[(-S] (0.3-1.5 mM). If ATP is used, an ATP regenerating system comprising phosphocreatine and creatine kinase may be included. RecA and oligonucleotide are generally added at a molar ratio of about 0.1-3 (RecA to nucleotides). If the probe is double-stranded, it must first be denatured before RecA coating. Incubation is at room temperature or, preferably, 37° C., for 5-30 min. D-loop or triple strand structure formation involves adding RecA filaments to dsDNA and incubating, preferably at 37° C., for about 15 min-2 hrs. It is also possible to form RecA filaments and do homology searching in a single reaction vessel, i.e., to mix RecA with oligonucleotides and dsDNA at the same time. See, for example, Rigas et al., supra; Honigberg, S M, et al., Proc Natl Acad Sci USA 83:9586-9590 (1986); any of the Ferrin et al. publications (supra).

Oligonucleotide extension can be accomplished by any primer dependent DNA polymerase (see Goelet, P et al., U.S. Pat. Nos. 5,888,819 and 6,004,744)

In another preferred embodiment the present system employs:

(1) RecA;

(2) two specific oligonucleotides that may contain 5′ or internal labels for detection or immobilization, which oligonucleotides are complementary to opposite strands in the target DNA at the site of SNP or mutation such that the 3′ end of each oligonucleotide is complementary to the same allele of the mutation or SNP; and

(3) DNA polymerase and dNTPs for extension of annealed oligonucleotides, all or some of which dNTPs may be detectably labeled or contain adducts to allow immobilization.

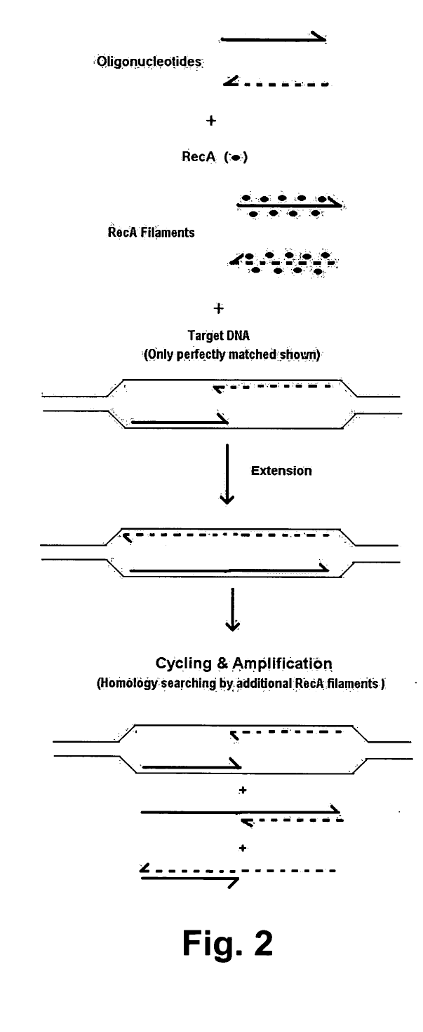

When oligonucleotide probes complementary to both strands of the target DNA are extended, double D-loops will be formed. Stable double D-loops are perfect targets for additional RecA mediated homology searching as are the double-stranded oligonucleotides displaced from double D-loops by homology searching (see FIG. 2). Therefore, RecA/ASOE assays using double D-loop formation can amplify exponentially.

The target DNA may be of any length (up to an entire chromosome) and can be either genomic or plasmid DNA or a PCR amplicon.

The detectably labeled oligonucleotides can be directly labeled with fluorophores or fluorescent labels, including, but not limited to, Fluorescein (and derivatives), 6-Fam, Hex, Tetramethylrhodamine, cyanine-5, CY-3, allophycocyanin, Lucifer yellow CF, Texas Red, Rhodamine, Tamra, Rox, Dabcyl. They may also be labeled with radioactive labels, digoxigenin, chemiluminescent labels or colorimetric labels.

RecA filament formation can be accomplished, for example, in a Tris-HCl or Tris-acetate buffer, (20-40 mM, pH 7.4-7.9) with MgCl2 or Mg acetate (1-4 mM), dithiothreitol (0.2-0.5 mM), and ATP or ATP[(-S] (0.3-1.5 mM). If ATP is used, an ATP regenerating system comprising phosphocreatine and creatine kinase may be included. RecA and oligonucleotide are generally added at a molar ratio of 0.1-3 (RecA to nucleotides). If the oligonucleotide is double-stranded, it must first be denatured before RecA coating. Incubation is at room temperature or, preferably, 37° C., for 5-30 min. D-loop or triple strand structure formation involves adding RecA filaments to dsDNA and incubating, preferably at 37° C., for about 15 min-2 hrs. It is also possible to form RecA filaments and do homology searching in a single reaction vessel, i.e., to mix RecA with oligonucleotides and dsDNA at the same time. See, for example, Rigas et al, supra; Honigberg, S M, et al, Proc Natl Acad Sci USA 83:9586-9590 (1986); any of the Ferrin et al. publications (supra).

Oligonucleotide extension can be detected by immobilizing the extended, detectably labeled oligonucleotides in an extension dependent fashion. For example, a dNTP may be bound to biotin to allow their immobilization to avidin or streptavidin coated surfaces, including but not limited to microtiter plates, magnetic beads and microspheres (beads). Alternatively, immobilization may be accomplished by allowing extended oligonucleotides to anneal to immobilized single stranded oligonucleotides (immobilization oligonucleotides) complementary to the extended sequence, i.e., not to the probe. Thus only following extension can probes be immobilized. When immobilization oligonucleotides are employed, immobilization of the oligonucleotides may be to microtiter plates, magnetic beads, beads suitable for detection via flow cytometry, microarrays or any other solid surface. Detection may be via any the methods well known in the art including, but not limited to, plate readers, flow cytometers and microarray readers.

In one preferred embodiment of this invention, RecA is mixed with a synthetic oligonucleotide, of any length, but preferably of 30-60 bases in length, under conditions that allow formation of RecA filament. Filament formation may occur before or after addition of oligonucleotide to double-stranded target DNA. Target DNA may be any dsDNA including, but not limited to, genomic DNA of any species, viral DNA, plasmid DNA, PCR amplicons, restriction fragments, or cloned DNA. The oligonucleotide is selected to be complementary to a specific region of the target DNA such that the 3′ end of the oligonucleotide complementary to one allele of the mutation or SNP to be detected.

Conditions are established, following formation of the RecA filament or following mixing of the RecA filament with target DNA, such that RecA filament is allowed to conduct a homology search on the target DNA. Provided complementary sequence exists in the target DNA, a triple stranded structure will be formed. This triple stranded structure will contain a 3′ end (of the oligonucleotide) suitable for extension by DNA polymerase. The DNA polymerase may be any polymerase and is not required to be thermostable.

Detection of extended oligonucleotides is accomplished by separating the extended oligonucleotides from oligonucleotides that have not been extended. This is preferentially accomplished by immobilizing the extended oligonucleotides, either by simply binding them to a solid support capable of binding DNA or by means of an adduct present in the dNTPs used for extension, such as biotin, or by annealing them to an oligonucleotide which has been immobilized to a solid support and which is complementary only to the extended sequence portion of the extended oligonucleotide. By using different detectable labels in the probe oligonucleotides, it is possible to score multiple alleles of a given mutation of SNP in a single reaction vessel. The complementary oligonucleotide method of immobilization allows multiplexing of the extension reaction to examine multiple sites in a single target DNA sample and yet score them separately. Alternatively, multiplexing can be accomplished by adding 5′ oligonucleotide “tails” to the extension oligonucleotides and detectably labeled dNTPs. In this case, different tails attached to extension oligonucleotides with 3′ ends complementary to different alleles will allow extension products to be scored independently.

Detection of label may be accomplished by a variety of methods including, but not limited to, plate readers capable of detecting visible or fluorescent signals, microarray readers and flow cytometers.

By allowing repeated formation of the triple stranded structure, preferably by performing homology searching in the presence of ATP, it is possible to have multiple oligonucleotides extended from each site in the target DNA without denaturation of the target DNA.

Efficient RecA-catalyzed D-loop formation, oligonucleotide extension and flow cytometric signal detection, (5,000-20,000 sequences are sufficient for a genotype determination) allows as many as 1000 or more separate assays to be performed on a single sample of blood.

This technology is ideally suited to multiplexing wherein several sites in a single sample of genomic, plasmid or amplified DNA are interrogated simultaneously. In this application, specific probes complementary to each allele of a mutation or SNP are designed with distinguishable labels and are used with unlabeled dNTPs or the extension oligonucleotides are designed with specific oligonucleotide tails and are used with labeled dNTPs. Extended oligonucleotides are specifically immobilized by use of immobilization oligonucleotides complementary to the extended sequence of each probe or to the specific oligonucleotide tails, respectively.

A major advantage of the RecA/ASOE SNP, mutation and specific sequence detection technologies is that they can operate on genomic DNA without denaturation or amplification.

It is difficult to overstate the power of the RecA/ASOE method. It is rapid, works with small samples and can readily be adapted to clinical applications for diagnostic genotyping and mutation/SNP detection. Further, the precision of RecA mediated homology searching allows the extremely accurate detection of infectious agents in samples with vast excesses of heterologous DNA. Perhaps the most important distinguishing advantage of the present invention is its complete independence from DNA amplification (i.e., PCR).

Kits

The present invention is also directed to kit or reagent systems useful for practicing the methods described herein. Such kits will contain a reagent combination comprising the essential elements required to conduct an assay according to the methods disclosed herein. The reagent system is presented in a commercially packaged form, as a composition or admixture where the compatibility of the reagents will allow, in a test device configuration, or more typically as a test kit, i.e., a packaged combination of one or more containers, devices, or the like holding the necessary reagents, and usually including written instructions for the performance of assays. The kit of the present invention may include any configurations and compositions for performing the various assay formats described herein.

Kits containing RecA, oligonucleotides and, where applicable, reagents for detection of fluorescent, chemiluminescent, radioactive or colorimetric signals, are within the scope of this invention. In one embodiment, a kit of this invention designed to allow detection of specific mutations and/or polymorphisms or mutations and/or in specific sequences of target DNA, includes oligonucleotides or other probes specific for (a) selected mutations and/or (b) SNPs, or (c) specific region or regions of target DNA. The probes may be labeled as described above. The kits also include a plurality of containers of appropriate buffers and reagents.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method of detecting a mutation, a single nucleotide polymorphism (SNP) or a specific sequence in a target DNA molecule comprising:
(a) providing a ssDNA probe complementary to a specific region of the target DNA including the site of said mutation, SNP or specific nucleotide sequence, wherein the 3′ end of said probe is complementary to the specific sequence or to a specific allele of the mutation or SNP being detected;
(b) contacting the probe with a RecA protein or a homologue of RecA to form a RecA filament;
(c) contacting the RecA filament with the target DNA, thereby forming a three stranded DNA D-loop structure in the target DNA, which D-loop structure comprises the probe and the two strands of the target DNA;
(d) extending said probe using DNA polymerase and dNTPs or dNTP analogs; and
(e) detecting extension of said probe,
wherein extension of said probe is indicative of the presence in said target DNA of (i) the mutation, SNP or specific sequence; or (ii) the allele of the mutation or SNP.

2. The method of claim 1 wherein said probe is a synthetic oligonucleotide

3. The method of claim 1 wherein said probe is labeled with a fluorescent, radioactive, chemiluminescent, enzymatic, antigenic or colorimetric adduct.

4. The method of claim 1 wherein said probe is bonded to an adduct that allows immobilization of the probe before or after extension.

5. The method of claim 4 wherein the adduct is biotin or digoxigenin.

6. The method of claim 4 wherein the adduct is an oligonucleotide.

7. The method of claim 1 wherein the RecA protein is from E. coli.

8. The method of claim 1 wherein the detecting is by flow cytometry.

9. The method of claim 1, wherein the DNA D-loop structure is stabilized by the addition of SSB protein.

10. The method of claim 1 wherein the target DNA molecule is selected from the group comprising prokaryotic genomic DNA, eukaryotic genomic DNA, cDNA, viral DNA, plasmid DNA, and a DNA fragment amplified by PCR or by another amplification method.

11. The method of claim 1 or 2 wherein the oligonucleotide is selected from the group consisting of:
(a) a synthetic oligonucleotide;
(b) a recombinant oligonucleotide; and
(c) an oligonucleotide obtained by denaturing, and optionally cleaving, a double-stranded DNA molecule.

12. The method of claim 11, wherein the oligonucleotide has a length of about 30 to about 80 nucleotides.

13. A kit useful for detecting a one or more mutations or SNPs in a target DNA sample, the kit being adapted to receive therein one or more containers, the kit comprising:
(a) RecA protein or a homologue of RecA;
(b) a specific oligonucleotide whose 3′ end is complementary to a region of the target DNA, adjacent to the mutation or SNP of interest, such that the base at the 3′ end of the oligonucleotide is complementary to one allele of the mutation or SNP; and, optionally
(c) DNA polymerase.

14. A kit useful for detecting a specific DNA sequence in a double-stranded DNA sample, the kit being adapted to receive therein one or more containers, the kit comprising:
(a) RecA protein or a homologue of RecA;
(b) a specific oligonucleotide complementary some portion of said specific DNA sequence; and optionally,
(c) DNA polymerase.

15. A kit according to claim 13 or 14 wherein said specific oligonucleotide contains a 5′ adduct to allow immobilization.

16. A kit according to claim 13 or 14 wherein said DNA polymerase is E. coli DNA polymerase.

17. A kit according to claim 13 or 14 wherein said DNA polymerase is a thermostable DNA polymerase.

18. A kit according to claim 13 or 14 wherein said oligonucleotide is labeled with a fluorescent, radioactive, chemiluminescent, enzymatic, antigenic or colorimetric label.
